(19) United States
(12) Patent Application Publication
(10) Pub. No.: US 2024/0153633 A1
(43) Pub. Date: May 9, 2024

(54) CLINICAL DIAGNOSTIC AND PATIENT INFORMATION SYSTEMS AND METHODS

(71) Applicant: IDEXX Laboratories, Inc., Westbrook, ME (US)

(72) Inventor: Raghavan PALANIAPPAN, Cape Elizabeth, ME (US)

(21) Appl. No.: 18/382,700

(22) Filed: Oct. 23, 2023

Related U.S. Application Data
(60) Provisional application No. 63/422,081, filed on Nov. 3, 2022.

(51) Int. Cl. G16H 50/20; G06N 3/088; G06N 3/09; G16H 50/70

(52) U.S. Cl. CPC G16H 50/20; G06N 3/088; G06N 3/09; G16H 50/70

(57) ABSTRACT

A clinical diagnostic system for predicting disease diagnosis from animal patient data is described. The system includes instructions for training first stage and second stage machine learning models for different species and breeds of animals. The first stage machine learning model is trained on unstructured data in the animal patient data to extract structured data. The extracted structured data is combined with other structured data included in the animal patient data to train one or more second stage machine learning models. The trained first and second stage machine learning models are applied, in sequence, on new patient medical record data to predict disease diagnosis.

### TECHNICAL FIELD

The present technology is generally related to the field of providing programmatic clinical decision support and more particularly to generating machine learning models and artificially intelligent systems for non-human patient diagnoses and for supporting clinical decisions in the veterinary space.

### CROSS REFERENCE TO RELATED APPLICATIONS

This application claims the priority benefit of U.S. Provisional Application No. 63/422,081, filed Nov. 3, 2022, the entire disclosure of which is hereby incorporated herein in its entirety by reference.

### BACKGROUND

The veterinary team is responsible for generating and managing an accurate and complete medical record of an animal patient's medical history, diagnosis, treatment, and care. The medical record can include clinical data gathered during patient visits to the veterinarian. Examples of such clinical data include demographic information (species, breed, gender etc.), vital signs, diagnoses, medications, treatment plans, progress notes, patient problems, vaccines, laboratory results, and radiographs, among others.

A medical record, health record or medical chart is a systematic documentation of a patient's medical history, test results, and care. A medical record may include one or both of a physical folder and an electronic folder for each individual patient and contains the body of information that comprises the total of each patient's health history. Medical history of a patient may be gained by a veterinarian or other healthcare professional by asking specific questions of people (for example, the pet owner) who know the patient and can give suitable information, with the aim of obtaining information useful in formulating a diagnosis and providing medical care to the non-human patient. The medical history can also include information on symptoms, laboratory test results, diagnoses, and treatment for each visit.

Conventionally, a veterinarian or other health care professional uses a combination of the patient's medical record and the present symptoms to generate a diagnosis. However, this diagnosis may be incorrect due to errors arising from both perceptual and system-related causes. Some common factors that lead to errors include misjudging the significance of observations, misinterpretation of test results, errors originating from heuristics usage, and errors in judgment, particularly when diagnostic hypotheses are developed and assessed. Since treatment options are becoming efficient and inexpensive, the health and financial risk of misdiagnosing an easily curable illness is significantly greater. Thus, there is a loss in improved patient care.

These diagnostic errors could be minimized using machine learning (ML) techniques to improve healthcare services. The kind of analytics a veterinarian can get using ML, at the time of patient treatment, can provide them with more knowledge and, thus, better care. ML algorithms have the ability to efficiently process large data sets, far beyond human limits, into clinical knowledge that enables veterinarians to prepare and deliver treatment, eventually leading to improved results, and lower medical costs.

However, the existing ML diagnostic systems are inadequate to handle animal patient data, which has much larger variability across animal species, breed, gender, and geographic location. Further, the medical data associated with animal patients includes a large amount of unstructured data that does not lend itself to traditional machine learning models. Thus, there is a need for machine learning algorithms, that go beyond the capabilities of the human mind, which large very large amounts of data across animal species, breed, gender, and geographic location, to provide diagnostic information.

### SUMMARY

At least the above-discussed need is addressed and technical solutions are achieved in the art by various embodiments disclosed in the present disclosure. As one example, non-human patient centric machine learning systems for non-human patient diagnoses and for supporting clinical decisions in the veterinary space are provided herein. Some embodiments pertain to a method for predicting diseases in animals. The method comprises receiving patient medical record data; filtering the received patient medical record data by at least one of species, breed, gender, or geographic location; separating the filtered patient medical record data into first structured data and unstructured data; training a first machine learning model on the unstructured data to extract second structured data from the unstructured data; combining the first structured data and the second structured data to form a training set for a second machine learning model; training the second machine learning model on the training set formed from the combined structured data to output the likelihood of one or more diseases; and applying the trained first machine learning model and the trained second machine learning model, in sequence, on new patient medical record data to predict disease diagnosis.

In some embodiments, the training set for the second machine learning model includes one or more input features extracted from the combined structured data and corresponding ground truth. In some embodiments, the one or more features include one or more of an age of the patient, propensity of the patient to one or more diseases, one or more test results, one or more symptoms, and one or more observations, and the ground truth includes the likelihood of one or more diseases.

In some embodiments, the first machine learning model is trained using unsupervised learning. In some embodiments, the second machine learning model is trained using supervised learning.

In some embodiments, the method further includes training a plurality of second machine learning models; evaluating the plurality of second machine learning models using one or more metrics; and selecting one or more machine learning models of the plurality of second machine learning models for application to the new patient medical record data to predict disease diagnosis.

In some embodiments, the one or more metrics include prediction error, complexity, explainability, or data size.

In some embodiments, the first structured data and the second structured data is combined based on data or time information included in the patient medical record data.

In some embodiments, a clinical diagnostic system comprises at least one computer accessible-storage device configured to store instructions corresponding to the method embodiments discussed above; and at least one processor communicatively connected to the at least one computer accessible storage device and configured to execute the instructions.

In some embodiments, a non-transitory computer readable storage medium configured to store a program, executed by a computer, for a clinical diagnostic system, the program including instructions corresponding to the method embodiments discussed above.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 shows a computing device system, according to some embodiments.

FIG. 2 shows another computing device system, according to some embodiments.

FIG. 3 shows a block diagram of a clinical diagnostic system, according to some embodiments; and

FIG. 4 shows a flowchart of a machine learning based disease prediction method, according to some embodiments.

### DETAILED DESCRIPTION

In some embodiments, the computer systems described herein execute methods for generating clinical diagnostic models from very large data sets using machine learning. It should be noted that the disclosure and claims are not limited to these embodiments, or any other examples provided herein, which are referred to for purposes of illustration only.

In this regard, in the descriptions herein, certain specific details are set forth to provide a thorough understanding of various embodiments of the disclosure and claims. However, one skilled in the art will understand that the disclosure and claims may be practiced at a more general level without one or more of these details. In other instances, well-known structures have not been shown or described in detail to avoid unnecessarily obscuring descriptions of various embodiments of the disclosure and claims.

Any reference throughout this specification to “one embodiment”, “an embodiment”, “an example embodiment”, “an illustrated embodiment”, “a particular embodiment”, “one aspect”, “an aspect”, “an example aspect”, “an illustrated aspect”, “a particular aspect” and the like means that a particular feature, structure or characteristic described in connection with the embodiment is included in at least one embodiment or aspect. The phrases embodiment and aspect may be used interchangeably in the disclosure. Thus, any appearance of the phrase “in one embodiment”, “in an embodiment”, “in an example embodiment”, “in this illustrated embodiment”, “in this particular embodiment”, or the like in this specification is not necessarily all referring to one embodiment or aspect, or a same embodiment or aspect. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, some embodiments may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

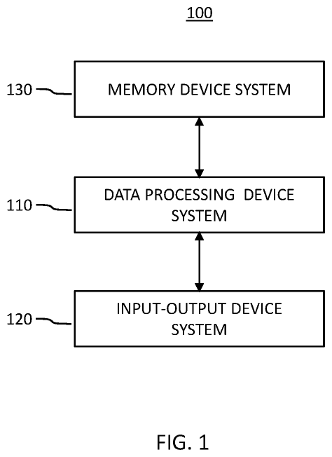

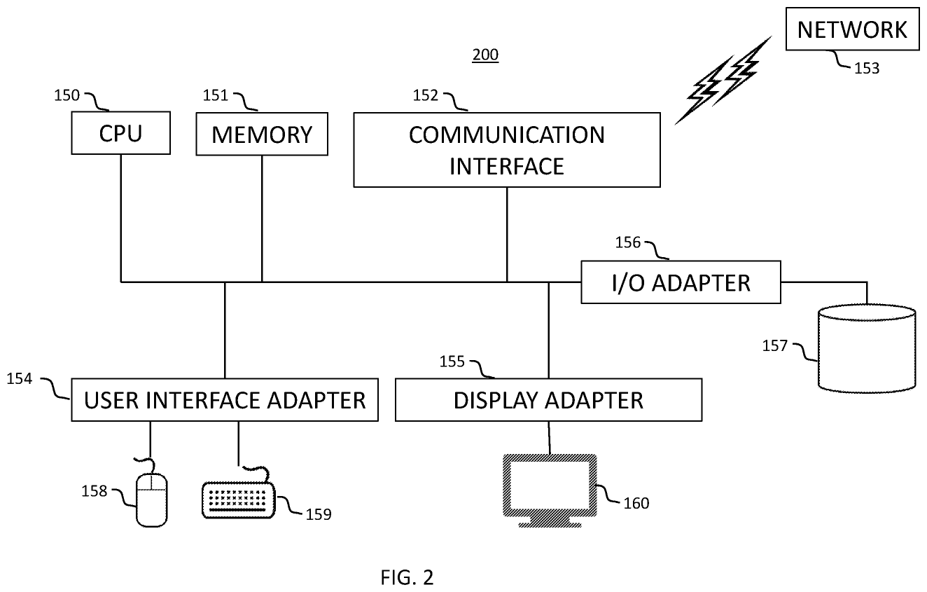

The term “program” in this disclosure should be interpreted to include one or more programs including as a set of instructions or modules that may be executed by one or more components in a system, such as a controller system or data processing device system, in order to cause the system to perform one or more operations. The set of instructions or modules may be stored by any kind of memory device, such as those described subsequently with respect to the memory device system 130, 151, or both, shown in FIGS. 1 and 2, respectively. In addition, this disclosure may describe or similarly describe that the instructions or modules of a program are configured to cause the performance of an action. The phrase “configured to” in this context is intended to include at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

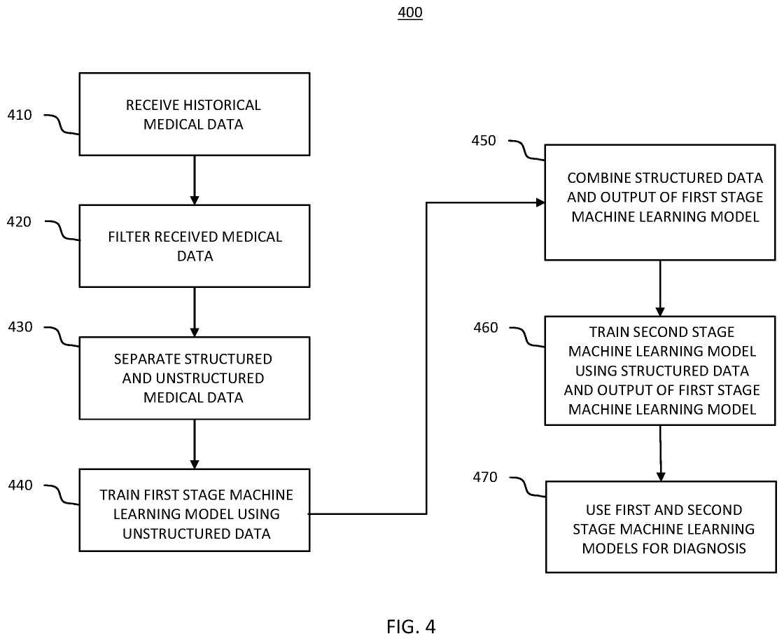

Further still, example methods are described herein with respect to FIG. 4. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangement shown in method 400 herein is not limited to an actual structure of any program or set of instructions or required ordering of method tasks, and such method 400, according to some embodiments, merely illustrates the tasks that instructions are configured to perform, for example upon execution by a data processing device system in conjunction with interactions with one or more other devices or device systems.

FIG. 1 schematically illustrates a system 100 according to some embodiments. In some embodiments, the system 100 may be a computing device 200 (as shown in FIG. 2). In some embodiments, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various embodiments. Each of the phrases “data processing device”, “data processor”, “processor”, and “computer” is intended to include any data processing device, such as a central processing unit (“CPU”), a desktop computer, a laptop computer, a mainframe computer, a tablet computer, a personal digital assistant, a cellular phone, and any other device configured to process data, manage data, or handle data, whether implemented with electrical, magnetic, optical, biological components, or other.

The memory device system 130 includes one or more processor-accessible memory devices configured to store information, including the information needed to execute the control programs associated with some of the various embodiments. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some embodiments, each of the phrases “processor-accessible memory” and “processor-accessible memory device” is intended to include a non-transitory computer-readable storage medium. In some embodiments, the memory device system 130 can be considered a non-transitory computer-readable storage medium system.

The phrase “communicatively connected” is intended to include any type of connection, whether wired or wireless, between devices, data processors, or programs in which data may be communicated. Further, the phrase “communicatively connected” is intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the embodiments.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the embodiments. In this regard, the input-output device system may include various other devices or systems described in various embodiments.

FIG. 2 shows an example of a computing device system 200, according to some embodiments. The computing device system 200 may include a processor 150, corresponding to the data processing device system 110 of FIG. 1, in some embodiments. The memory 151, input/output (I/O) adapter 156, and non-transitory storage medium 157 may correspond to the memory device system 130 of FIG. 1, according to some embodiments. The user interface adapter 154, mouse 158, keyboard 159, display adapter 155, and display 160 may correspond to the input-output device system 120 of FIG. 1, according to some embodiments. The computing device 200 may also include a communication interface 152 that connects to a network 153 for communicating with other computing devices 200.

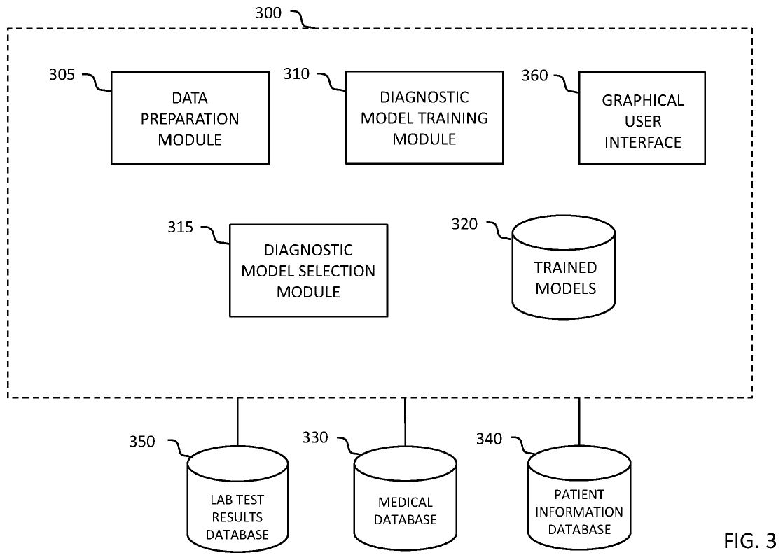

Various methods 400 may be performed by way of associated computer-executable instructions according to some example embodiments. In various example embodiments, a memory device system (e.g., memory device system 130) is communicatively connected to a data processing device system (e.g., data processing device systems 110, otherwise stated herein as “e.g., 110”) and stores a program executable by the data processing device system to cause the data processing device system to execute various embodiments of methods 400 via interaction with at least, for example, various databases 320, 330, 340, 350 shown in FIG. 3. In these various embodiments, the program may include instructions configured to perform, or cause to be performed, various ones of the instructions associated with execution of various embodiments of methods 400. In some embodiments, methods 400 may include a subset of the associated blocks or additional blocks than those shown in FIG. 4. In some embodiments, methods 400 may include a different sequence indicated between various ones of the associated blocks shown in FIG. 4.

FIG. 3 shows an example of a clinical diagnostic system 300, according to some embodiments. According to some embodiments, the systems 100, 200 provide some or all of the system 300 shown in FIG. 3. In this regard, FIG. 3 illustrates a system 300, according to some embodiments. The system 300 may be a particular implementation of the systems 100, 200 according to some embodiments. In some embodiments, the clinical diagnostic system 300 is implemented by programmed instructions stored in one or more memories and executed by one or more processors of the systems 100, 200.

In some embodiments, the clinical diagnostic system 300 includes a data preparation module 305, a diagnostic model training module 310, a diagnostic model validation/selection module 315, and one or more diagnostic models 320. In some embodiments, the clinical diagnostic system 300 may be communicatively connected to one or more databases 330, 340, and 350. In some embodiments, the clinical diagnostic system 300 includes a graphical user interface 360 to permit a user to interact with the system. In some embodiments, the one or more diagnostic models 320 may be stored in a database.

In some embodiments, a medical database 330 stores reference medical data such as ranges of normal, low, and high test results for various diagnostic tests performed by the one or more veterinary laboratories or one or more diagnostic testing instruments. In some embodiments, the diagnostic tests may be performed by mobile laboratories, using home testing kits, etc. In some embodiments, the computing device system 200 may access the medical database 330 to compare actual patient test results, stored in laboratory test results database 350, with the typical ranges stored in the medical database to interpret the test results from the one or more veterinary laboratories or the one or more diagnostic testing instruments. In some embodiments, the diagnostic tests include blood chemistry and PCR assays.

In some embodiments, a patient information database 340 stores a patient's medical record, including patient demographic information, vital signs at each visit, diagnoses, medications, treatment plans, progress notes, patient problems, vaccine history, test results, and imaging data such as radiographs. The demographic data may include species, breed, weight, age, gender, and geographic location, for example. In some embodiments, the medical record may also include information on test results (blood chemistry, pathology, and PCR (polymerase chain reaction) panels/assays), vector of exposure, and diagnoses, obtained from the laboratory test results database 350. Blood chemistry tests may include results for hemogram, five-part differential, platelets, platelet indices, reticulocyte count, reticulocyte hemoglobin, abnormal red and white blood cell morphology, blood parasites, unclassified cells, and immature cell lines, among others.

Polymerase chain reaction (PCR) testing identifies the presence of a pathogen's DNA or RNA in a patient specimen. PCR is a highly sensitive and specific testing that can confirm the actual presence of an organism and facilitate early detection of disease in sick animals. PCR panel results are often positive in infected animals before antibodies can be detected, providing early indication of disease. PCR panels commonly performed, based on presenting symptoms, include ringworm (Dermatophyte) panel, respiratory disease panels, H3N2 canine influenza virus test, diarrhea panel, vector-borne disease panel, canine distemper virus (CDV) quant test, Leptospira test, feline infectious peritonitis virus (FIPV) test, and feline enteric coronavirus (FECV) test, among others.

While the data in the medical database 330 is generally stored as structured data, information in the medical record 340 may be stored as both structured data and unstructured data.

Structured data is comprised of clearly defined data types with patterns that make them easily searchable; while unstructured data—“everything else”—is comprised of data that is usually not as easily searchable, including formats like audio, video, and free-form text (for example, clinical visit notes). Structured data usually resides in relational databases (RDBMS). RDBMS fields store variable length data like breed, geographic location, age, gender, and diagnosis. RDBMS fields can also store one or more paired data such as laboratory test type and result. It is a simple matter to search and use structured data in automated clinical support systems. Structured data may be human- or machine-generated, as long as the data is created within an RDBMS structure. This format is eminently searchable, both with human-generated queries and via algorithms using types of data and field names.

Unstructured data is essentially everything else. Unstructured data has an internal structure but is not structured via predefined data models or schema. It may be textual or non-textual, and human- or machine-generated. It may also be stored within a non-relational database. The most common type of unstructured data in veterinarian clinical diagnostic systems is patient notes and diagnostic images such as x-rays or CT scans. Mature analytics tools exist for structured data, but analytics tools for mining unstructured data are nascent and developing. The lack of orderly internal structure defeats the purpose of traditional data mining tools. One option for extracting information from unstructured text data is using predetermined keywords or key phrases. However, this approach prevents the diagnostic system from learning new or unknown information present in the unstructured text and does not account for variation in how different veterinarians may refer to or record the same information.

Working with images is even more difficult than working with unstructured text. Images may be of different sizes, stored in different formats, and captured under different conditions. Thus, before being used to train models, images have to undergo a set of transformations to normalize the images across the entire dataset. Computer vision, understanding and extracting the unstructured information stored in images, generally requires AI-based deep learning models to analyze images with results that surpass human-level accuracy. The field of computer vision includes a set of main problems such as image classification, localization, image segmentation, and object detection. Among those, image classification can be considered the fundamental problem. It forms the basis for other computer vision problems.

Image classification is the task of categorizing and assigning labels to groups of pixels or vectors within an image depending on particular rules. The labeled data can be stored as structural data to be used for training other machine learning models. In some embodiments, the data preparation module 305 includes programmed instructions to generate machine learning models for extracting structured data from unstructured text and images included in the medical records.

In some embodiments, a lab test results database 350 stores diagnostic test information. The lab results database 350 may also store associated information including symptoms for various diseases and follow-on testing performed in each situation.

In some embodiments, the data preparation module 305 communicates with the one or more databases 330, 340, 350 to receive medical record and medical history data for a large population of patients, gathered over a period of time.

In some embodiments, the medical record includes biographical information, such as age, gender, breed, and geographic location, one or more of which may be used as input features in training the machine learning models. These features can provide contextual discriminatory power to the machine learning models—such as variations in disease presentation (and factors) because of differences in gender or breed type. Moreover, geographic location may provide important environmental factors that impact the likelihood of various types of diseases. For example, a dog that lives in a highly urbanized area is much less likely to get Lyme disease than a dog that lives in a forested area.

In addition to biographical information, the medical record and the medical history data include information on any diagnostic tests that have been performed, and patient notes entered by a veterinarian. For example, a complete blood count (CBC) test includes data (results) for red blood cell count, hematocrit, hemoglobin, mean cell volume, mean corpuscular hemoglobin, mean corpuscular hemoglobin concentration, red blood cell redistribution width, reticulocytes (percentage and number), reticulocyte hemoglobin, nucleated red blood cells, white blood cell count, neutrophils (percentage and number), lymphocytes (percentage and number), monocytes (percentage and number), eosinophils (percentage and number), basophils (percentage and number), band neutrophils, platelet count, platelet distribution width, mean platelet volume, plateletcrit, total nucleated cell count, agranulocytes (percentage and number), and granulocytes (percentage and number). Other tests may check for presence, count, or concentration of squamous epithelial cells, non squamous epithelial cells, bacteria (rods or cocci), hyaline and nonhyaline casts, or crystals (bilirubin, ammonium biurate, struvite etc.). Levels of various proteins, enzymes, or minerals may have been checked. Other tests to check for specific pathogens may have been run, and their results stored in the medical record and the medical history data. The diagnosis of the veterinarian may be stored as structured data (for example, as check boxes on fields associated with various illnesses) or as unstructured data (for example, in the veterinarian's notes).

In some embodiments, the data preparation module 305 receives the medical record and medical history data, and extracts features to be used as inputs for training machine learning models to classify various diseases. The ground truth (target output from the machine learning models) is provided by the diagnosis associated with the corresponding set of tests and observations in the patient's medical record and medical history data. In some embodiments, the medical tests are associated with the diagnosis based on dates, or links between the tests and the diagnosis. In some embodiments a filtering operation is performed to separate the data by species, breed, gender or even geographic location, to permit training of more specialized machine learning diagnostic models, that provide more discriminatory power than a generalized machine learning trained on data for all animal types and locations.

In some embodiments, the data preparation module 305 first separates the structured data from unstructured text included in the medical record and the medical history data, to be used as training data for the machine learning model. For example, the veterinarian's notes in the medical record may include information on the family history, symptoms exhibited by the patient, the veterinarian's observations, and the diagnosis. In some embodiments, text tagging and annotation is performed on the unstructured text in the medical data by identifying various terms or entities (for example, symptoms, test results, observations, medications, or diagnoses) recorded in the unstructured text using domain-specific ontologies. The input to the text tagging process is the unstructured text of the medical data and one or more ontologies; the output from the text tagging process is annotated semantic data (the extracted knowledge) that can be stored in structural data format. Text tagging and annotation consists of identifying the occurrence of terms or entities described in the ontologies in the freeform or unstructured text.

In some embodiments, the text tagging and annotation process may be performed using forms and templates, which record semi-structured data. In these embodiments, various fields in the forms and templates define the ontologies or the structured data fields. For example, for each patient visit, the veterinarian may record the vital signs of the patient in various structured fields. The patient visit form may also include a list of symptoms that the veterinarian can select. The patient record form also contains a notes field section where the veterinarian can record their findings, diagnoses, and treatment plans. This notes field is where information is entered as natural language text or freeform text, and this unstructured data is key to building machine learning models for automatically predicting various diseases based on symptoms, observations, and test results, especially since the structured fields may not always be complete or accurate. For example, different users often don't follow the same procedures or standard protocols to record the data in the patient's electronic record.

It is obvious that similar data may be stored as either structured data or unstructured data in the medical record and medical history. For example, the medical record filled out by a veterinarian during a patient visit may include structured fields corresponding to various symptoms such as fever, diarrhea, or jaundiced eyes. However, the veterinarian may or may not use the structured fields to record this data and instead type the symptoms in the unstructured notes field.

Domain ontologies are helpful to extracting the information recorded in the unstructured text. The domain ontologies describing the information recorded in the unstructured text, and the relationships between the different types of information recorded in the unstructured text, may be prepared by domain experts or automatically learned from the unstructured data using unsupervised machine learning techniques. Using domain experts to prepare ontologies is a costly and time-consuming effort. The expert must determine the scope of the ontology based on what the ontology is going to be used for, who will use and maintain it, and what types of knowledge needs to be extracted using the ontology. More importantly, the ontology's performance and accuracy will be limited both by the domain expert's knowledge and assumptions, and by a user's proper use of the ontology. Even in cases where a domain expert defines a good ontology, the veterinarians may not use the ontology when recording their notes. Different veterinarians may use different terms for the same concept. Accordingly, in order to resolve these drawbacks of domain expert based ontologies, in some embodiments, unsupervised machine learning techniques are used to automatically generate and update ontologies for extracting structured knowledge from the unstructured notes data stored in the medical records and the medical history information.

In some embodiments, a segmentation process is performed on the unstructured data fields, such as the notes field in the patient visit record, to identify the starting and ending boundaries of the text phrases and words present in the medical records. The text snippets are pre-processed by performing techniques such as, but not limited to, word frequency counting, dependency parsing, context tracing, and part-of-speech tagging. For example, word frequency counting identifies the most commonly occurring words and phrases in the unstructured text. Context tracing and part-of-speech tagging are used to identify the salient semantic-based words and phrases in the set of most commonly occurring words. Dependency parsing identifies the relationships between the words or phrases to determine the grammatical structure of a sentence. It is obvious to one of ordinary skill in the art that different veterinarians may use different words or phrases to describe the same entity or concept, and the grammar or contextual arrangement in their sentences may differ. Thus, in some embodiments, unsupervised learning on the extracted set of words and phrases is performed to find the associations, relations, and normalizations within the set of words and phrases.

Unsupervised learning essentially means that the data is not tagged with ground truth (the desired output class). Thus, the machine learning model is not trying to learn how to classify input features in an output class (such as the terms in a domain expert defined ontology) but, rather, the patterns present in the data. Unsupervised learning permits the data extraction process to efficiently extract the information present in the unstructured fields of the medical records and store it as a compact set of structured data (knowledge) to be used for further classification and use.

In some embodiments, a first stage machine learning model, for example a convolution neural network, is trained and used to automatically extract an ontology from the set of words and phrases in the medical records or medical history data. In the training phase, the untagged set of words and phrases extracted from the medical records or medical history data are provided as training data to one or more neural network models as inputs. The neural network models try to mimic the data they are given, and use the error in their mimicked output to correct themselves (that is, correct the weights and biases for each connected pair of neurons) by adjusting their parameters as more data is input. The error may be expressed as a low probability that erroneous output occurs, or as an unstable high energy state in the neural network. After training is completed, the neural network models output a “reference set” of concepts (ontology) that summarizes the set of words and phrases extracted from the medical records or medical history data. In other words, the neural network models self-learn the associations and relations present in the set of words and phrases, and output a reduced normalized set of concepts that capture the associations and relations present in the set of words and phrases.

In some embodiments, validation and testing of the trained first stage machine learning model is performed to ensure that the model is generalized (it is not overfitted to the training data and can provided similar performance on new data as on the training data). In some embodiments, a portion of the data is held back from the training set for validation and testing. The validation dataset is used to estimate the neural network's performance while tuning the neural network's parameters (weights and biases). The test dataset is used to give an unbiased estimate of the performance of the final tuned neural network model. It is well known that evaluating the learned neural network model using the training set would result in a biased score as the trained model is, by design, built to learn the biases in the training set. Thus, to evaluate the performance of a trained machine learning model, one needs to use data that has not been used for training.

In one embodiment, the collected data set of words and phrases extracted from the unstructured text fields of the medical records or medical history data can be divided equally between the training set and the testing set. The neural network models are trained using the training set and their performance is evaluated using the testing set. The best performing neural network model may be selected for use. The neural network model is considered to be generalized or well-trained if its performance on the testing set is within a desired range (error) of the performance on the training set. If the performance on the test set is worse than the training set (the difference in error between the training set and the testing set is greater than a predefined threshold), a two-stage validation and testing approach may be used.

In some embodiments, in a two stage validation and testing approach, the collected data set of words and phrases extracted from the maintenance records is divided between the training set, the validation set, and the testing set. The neural network models are first trained using the training set, then their parameters are adjusted to improve their generalization using the validation set, and, finally, the trained neural network models are tested using the testing set.

In some embodiments, the data set may be divided equally between the desired training, validation, or testing sets. This works well when there is a large collection of data to draw from. In cases where the collection of data samples is limited, other well known techniques, such as leave one out cross validation and testing or k-fold cross validation may be used to perform validation and testing. Cross-validation is a resampling procedure used to evaluate machine learning models on a limited data sample. The procedure has a single parameter called k that refers to the number of groups that a given data set is to be split into. As such, the procedure is often called k-fold cross-validation. When a specific value for k is chosen, such as k=10, the procedure becomes 10-fold cross-validation.

Cross-validation is primarily used to estimate how the trained model is expected to perform in general when used to make predictions on data not used during the training of the model. The dataset is shuffled randomly and divided into a predefined number (k) of groups. The training and testing process is performed k times, with one of the groups of data being held out as the testing set for each iteration and the remaining k−1 groups being used as the training set. Each model is fitted (trained) on the training set and evaluated (tested) on the test set to determine the level of generalization of the trained models.

The purpose of k-fold cross validation is not to pick one of the trained models as the first stage machine learning model but, rather, to help determine the model structure and the parameter training process for the first stage machine learning model. For example, a neural network model can have one or more “hidden” layers of neurons between the input layer and the output layer. Further, different neural network models can be built with different numbers of neurons in the hidden layers and the output layers. In some embodiments, in the training phase, a plurality of neural network models having different numbers of layers and different numbers of neurons in each layer are generated. Each of the plurality of neural network models is trained using k-fold cross validation, resulting in a score that predicts the skill of each model in extracting the set of concepts that capture the associations and relations present in the set of words and phrases in unseen (future) data. The model (number of layers and number of neurons in each layer) having the highest predictive score is selected and then trained on the entire data set of words and phrases present in the maintenance records to generate the final first stage machine learning model for extracting the knowledge stored in the unstructured data of the medical records or medical history.

It is obvious to one of ordinary skill in the art that the unsupervised first stage machine learning model is not limited to neural networks and other machine learning models, such as a Markov random field network, support vector machine, random forest of decision trees, or k-nearest neighbor, or a combination of different types of machine learning models may be used to extract the set of concepts that capture the associations and relations present in the medical records or medical history data.

In some embodiments, the extracted concepts from medical records or medical history data are stored as related structural data and used, together with the structural data present in the medical records or medical history data, to predict the likelihood of various diseases using a second stage machine learning model, discussed below.

In some embodiments, one or more second stage machine learning models are trained on the output (the knowledge extracted from the medical records or medical history data) from the first stage machine learning model and the structured data present in the medical records or medical history data to predict the likelihood of various diseases. In the training phase for the second stage machine learning models, the output from the first stage machine learning model and the other structured data present in the medical records or medical history data are provided as training data to the one or more second stage machine learning models as inputs. Some examples of input features derived from the first stage machine learning model include symptoms (such as “red eyes”, “loss of appetite”) and clinical observations (such as “blood pressure”, “temperature”, “lethargy”, “decreased response to stimuli”). Some examples of input features derived from the other structured data present in the medical records or medical history data include vital signs, test results, symptoms etc.

The second stage machine learning models are trained using supervised learning because the medical records or medical history data includes ground truth information on the veterinarian's diagnosis for each patient visit. Thus, each input feature vector has a target output label. The second stage machine learning models are trained to detect the underlying patterns and relationships between the input data and the output labels, enabling the models to yield accurate labeling results when presented with never-before-seen data.

Thus, the error functions used for unsupervised learning of the first stage machine learning models are different from the error functions used for supervised learning of the second stage machine learning models. For example, backpropagation may be used to train one or more neural networks models as second stage machine learning models using supervised learning. The backpropagation algorithm looks for the minimum value of the error function in weight space using a well known technique called the delta rule or gradient descent. The weights that minimize the error function are then considered to be a solution to the learning problem. The backpropagation approach works better at finding the optimal model, without overfitting, than merely reducing the error between the target output labels and the actual output labels (from the trained model). In backpropagation, the weights and biases are repeatedly adjusted forward (increased) or backwards (decreased) for each layer of the neural network, starting with the output layer and working back to the input layer, in an effort to find the global minimum of the error function. Each backward propagation iteration uses the error from a forward computation of the neural network in a previous iteration as the starting point for the adjustments. If the error has increased between iterations, the weights are adjusted in an opposite direction (if increasing weights increases the errors, then weights are decreased).

In another example, Euclidean distance may be used to train one or more k-means clustering classifiers as second stage machine learning models using supervised learning. In k-means clustering, the input feature vectors are partitioned into k clusters in which each observation belongs to the cluster with the nearest mean (cluster centers or cluster centroid), serving as a prototype of the cluster. The clusters correspond to the various diseases (ground truth). The most common algorithm (naïve k-means clustering) uses an iterative refinement technique. Given an initial set of k means (associated with k clusters), each input feature vector (collection of input features corresponding to one clinical visit, for example) is assigned to the cluster whose mean value is closest of the input feature vector in n-dimensional space (where n is the number of data points in the input feature vector). Various well known error functions, such as Euclidean distance or least squared Euclidean distance, may be used to make the assignment. After each assignment, the mean for each cluster is recalculated (since each assignment of an input feature vector changes the mean of the cluster to which it is assigned). After updating the mean values of each cluster, the vector reassignment is again performed. As the mean values of the clusters have changed, some feature vectors may now be closer to another cluster mean instead of a previously assigned cluster. The process of iteratively assigning feature vectors to various clusters and recomputing the means is performed repeatedly until convergence—the feature vectors do not change their assignment after recomputation of the means. The error function captures the number of feature vectors incorrectly assigned to a cluster (for example, an input feature vector corresponding to a diagnosis of heart disease being assigned to a cluster of feature vectors that are associated with lung disease. It is easy to understand why this problem is computationally hard—training a k-means clustering algorithm requires determined both the number of k means to partition the data into and the convergence.

In mathematical terms, the k-means clustering problem is NP-hard, which means there is no polynomial time algorithm to solve the problem. In other words, a human being would never be able to solve this problem, even if given an infinite amount of time. In machine learning, a variety of well-known heuristic algorithms are used to estimate a near optimal partitioning of the input feature vectors.

It is obvious to one of ordinary skill in the art that other well known machine learning techniques, such as k-nearest neighbor classifier and support vector machines, or a mix thereof, may be used to build the one or more second stage machine learning models.

In some embodiments, validation and testing of the trained second stage machine learning models is performed to ensure that the models are generalized (they are not overfitted to the training data and can provided similar performance on new data as on the training data). The validation and testing methods for the second stage machine learning model are similar to those of the first stage machine learning model. That is, validation and testing of the second stage machine learning models may be performed using well known techniques such as k-fold cross validation discussed in detail above.

The final second stage machine learning models may be selected from the trained one or more second stage machine learning models using various criteria. While it may seem obvious to select the machine learning model with the best performance (lowest error on test data), there are other considerations that may impact the choice of the final model. The quality of the model's results is a fundamental factor to consider when choosing a model. While algorithms that maximize performance could be prioritized, different metrics may be useful to analyze the results of the model. For example, model accuracy is not appropriate when working with imbalanced datasets. Selecting a good metric (or set of metrics) to evaluate the machine learning model's performance is a crucial task before selecting the model.

In many situations, explaining the results of a machine learning model is paramount. This is especially true in clinical diagnostic environments, where understanding the relationship between the input features (symptoms, observations, and test results) and the predict diseased diagnosis is necessary to convince the veterinarian, and the patient's caretakers, on a course of treatment. Unfortunately, many machine learning algorithms work like black boxes, and the results are hard to explain, regardless of how good they are. Linear regression models and decision trees are good candidates when explainability is an issue, neural networks are not.

A complex model can find more interesting patterns in the data, but at the same time, it will be harder to maintain and explain. More complexity can lead to better performance but also larger costs. Complexity is inversely proportional to explainability. The more complex the model is, the harder it will be to explain its results. Putting explainability aside, the cost of building and maintaining a model is a crucial factor for a successful project. A complex setup will have an increasing impact during the entire lifecycle of a model. The amount of training data available is also an important factor to consider when choosing a model. While a neural network is really good at processing and synthesizing tons of data, k-nearest neighbors classifier is much better with fewer examples.

In some embodiments, a combination of performance, explainability, and complexity is used to select the final machine learning model to predict disease diagnoses.

In some embodiments, separate second stage machine learning models may be built for each disease type. In these embodiments, the set of training data is grouped by disease type, and the appropriate group of data, extracted from the medical records and the medical history data, is used to train each second stage machine learning model. In some embodiments, the input features for training a kidney disease prediction model include “creatinine levels”, “glucose levels”, “SDMA (Symmetric dimethylarginine)”, “BUN (Blood Urea Nitrogen)”, “urine sugar”, “urine specific gravity”, “urine proteinuria”, “urine creating albumin ration” and other features extracted from PCR/Immunoassays, biopsies, ultrasounds, and MRI or CT scans. In some embodiments, the input features for training a liver disease prediction model include “ALT (Alanine Transaminase)”, “AST (Aspartate transaminase)”, “alkaline phosphate levels”, “CRP (C-Reactive protein)”, and other features extracted from PCR/Immunoassays, biopsies, ultrasounds, and MRI or CT scans. In some embodiments, similar input features could be utilized for other diseases, such as heart disease.

It is obvious to one of ordinary skill in the art that the machine learning models for the second stage classifier are not limited to neural networks and other machine learning models, such as k-means clustering, k-nearest neighbors, linear regression, support vector machine, random forest of decision trees, or a combination of different types of machine learning models may be used to predict the probability or likelihood of a particular disease.

In some embodiments, the trained second stage machine learning models are used to predict the likelihood of various diseases based on new data, and provide a “second read” to the veterinarian's diagnosis. In some embodiments, the veterinarian uses the second stage machine learning models to further inform their diagnosis. The veterinarian has the choice to use the trained machine learning models before or after they have made their diagnosis. Thus, the machine learning provide both guidance and confirmation of the diagnosis.

FIG. 4 shows a flowchart of a method 400 of training machine learning models to predict disease likelihood in animals. In step 410, medical record and the medical history data for a large population of patients is received. In step 420, the received data is filtered by one or more criteria to extract the relevant set of data for training the machine learning models. The criteria includes, among others, species, breed, gender, geographic location, and age. In some embodiments, a combination of criteria, maybe used to extract specific data that can be used to train specialized machine learning models. For example, all dogs do not suffer from the same diseases, and the presentation of a particular disease in different breeds may include different symptoms or different values for test results. Thus, a machine learning model that is trained on medical data from just “golden retrievers”, for example, may outperform a general machine learning model trained on medical data from all dog breeds. In step 430, the received medical data in separated into structured data and unstructured data. Structured data usually corresponds to test results, and other information entered into predetermined fields by a veterinarian or another healthcare professional. Unstructured data usually corresponds to veterinarian notes in the patient's medical record and medical history.

In step 440, a first stage machine learning model is trained using the unstructured text in the received medical data as training data for the first machine learning model. The trained first stage machine learning model outputs a plurality of concepts corresponding to the information present in the unstructured text in the medical data. The plurality of concepts output from the trained machine learning model may be stored, for example, as second structured data. In step 450, the structured data present in the medical record and the plurality of concepts (second structured data) extracted as output from the first stage machine learning model are combined. In some embodiments, date and timestamp information is used to associate and group corresponding portions of the data. For example, the medical record for a particular patient visit may include several days—spanning pre-visit tests, day of visit medical record and notes, and post-visit tests. Information in the unstructured text, and date and timestamp information, can be used to determine which pre- and post-visit tests are associated with a particular day of visit medical record. Each associated set of data, corresponding to one or more clinical visits (if for the same illness), may then be used to generate an input feature vector using the information in the combined structured data. The diagnosed disease is used as ground truth for the input feature vector.

In step 460, a second stage machine learning model is trained using the input feature vectors extracted from the combined structured data. The second stage machine learning model outputs predicted disease diagnoses. The trained machine learning models may be stored in a database and applied in sequence, on new patient medical record data to predict disease diagnosis (step 470).

It is important to note that the machine learning and diagnostic algorithms described herein do not represent a computer application of the way humans perform diagnoses. Humans interpret new data in the context of everything else they have previously learned. In stark contrast to mental diagnostic processes, artificial intelligence algorithms, and specifically, the machine learning (ML) algorithms described herein, analyze massive data sets to identify patterns and correlations, without understanding any of the data they are processing. This process is fundamentally different from the mental process performed by a veterinarian. Furthermore, the large amounts of data required to the train the machine learning models, and the complexity of the trained models, make it impossible for the algorithms described herein to be performed merely in the human mind.

In a first aspect A1, the present disclosure provides a processor executed method for predicting diseases in animals, the method comprising receiving patient medical record data; filtering the received patient medical record data by at least one of species, breed, gender, or geographic location; separating the filtered patient medical record data into first structured data and unstructured data; training a first machine learning model on the unstructured data to extract second structured data from the unstructured data; combining the first structured data and the second structured data to form a training set for a second machine learning model; training the second machine learning model on the training set formed from the combined structured data to output the likelihood of one or more diseases; and applying the trained first machine learning model and the trained second machine learning model, in sequence, on new patient medical record data to predict disease diagnosis.

In a second aspect A2, the present disclosure provides the method according to aspect A1, wherein the training set for the second machine learning model includes one or more input features extracted from the combined structured data and corresponding ground truth.

In a third aspect A3, the present disclosure provides the method according to aspect A2, wherein the one or more features include one or more of an age of the patient, propensity of the patient to one or more diseases, one or more test results, one or more symptoms, and one or more observations, and wherein the ground truth includes the likelihood of one or more diseases.

In a fourth aspect A4, the present disclosure provides the method according to any one of aspects A1-A3, wherein the first machine learning model is trained using unsupervised learning.

In a fifth aspect A5, the present disclosure provides the method according to any of aspects A1-A4, wherein the second machine learning model is trained using supervised learning.

In a sixth aspect A6, the present disclosure provides the method according to any of aspects A1-A5, wherein the method further includes training a plurality of second machine learning models; evaluating the plurality of second machine learning models using one or more metrics; and selecting one or more machine learning models of the plurality of second machine learning models for application to the new patient medical record data to predict disease diagnosis.

In a seventh aspect A7, the present disclosure provides the method according to aspect A6, wherein the one or more metrics include prediction error, complexity, explainability, or data size.

In an eighth aspect A8, the present disclosure provides the method according to any of aspects A1-A7, wherein the first structured data and the second structured data is combined based on data or time information included in the patient medical record data.

In a ninth aspect A9, the present disclosure provides a clinical diagnostic system comprising at least one computer accessible-storage device configured to store instructions; and at least one processor communicatively connected to the at least one computer accessible storage device and configured to execute the instructions to: receive patient medical record data; filter the received patient medical record data by at least one of species, breed, gender, or geographic location; separate the filtered patient medical record data into first structured data and unstructured data; train a first machine learning model on the unstructured data to extract second structured data from the unstructured data; combine the first structured data and the second structured data to form a training set for a second machine learning model; train the second machine learning model on the training set formed from the combined structured data to output the likelihood of one or more diseases; and apply the trained first machine learning model and the trained second machine learning model, in sequence, on new patient medical record data to predict disease diagnosis.

In a tenth aspect A10, the present disclosure provides the system according to aspect A9, wherein the training set for the second machine learning model includes one or more input features extracted from the combined structured data and corresponding ground truth.

In an eleventh aspect A11, the present disclosure provides the system according to aspect A10, wherein the one or more features include one or more of an age of the patient, propensity of the patient to one or more diseases, one or more test results, one or more symptoms, and one or more observations, and wherein the ground truth includes the likelihood of one or more diseases.

In a twelfth aspect A12, the present disclosure provides the system according to any one of aspects A9-A11, wherein the first machine learning model is trained using unsupervised learning.

In a thirteenth aspect A13, the present disclosure provides the system according to any one of aspects A9-A12, wherein the second machine learning model is trained using supervised learning.

In a fourteenth aspect A14, the present disclosure provides the system according to any one of aspects A9-A13, wherein the at least one processor is further configured to execute the instructions to: train a plurality of second machine learning models; evaluate the plurality of second machine learning models using one or more metrics; and select one or more machine learning models of the plurality of second machine learning models for application to the new patient medical record data to predict disease diagnosis.

In a fifteenth aspect A15, the present disclosure provides the system according to aspect A14, wherein the one or more metrics include prediction error, complexity, explainability, or data size.

In a sixteenth aspect A16, the present disclosure provides the system according to any one of aspects A9-A15, wherein the first structured data and the second structured data is combined based on data or time information included in the patient medical record data.

In a seventeenth aspect A17, the present disclosure provides a non-transitory computer readable storage medium configured to store a program, executed by a computer, for a clinical diagnostic system, the program including instructions for: receiving patient medical record data; filtering the received patient medical record data by at least one of species, breed, gender, or geographic location; separating the filtered patient medical record data into first structured data and unstructured data; training a first machine learning model on the unstructured data to extract second structured data from the unstructured data; combining the first structured data and the second structured data to form a training set for a second machine learning model; training the second machine learning model on the training set formed from the combined structured data to output the likelihood of one or more diseases; and applying the trained first machine learning model and the trained second machine learning model, in sequence, on new patient medical record data to predict disease diagnosis. Subsets or combinations of various embodiments/aspects described above provide further embodiments.

These and other changes can be made to the disclosure and claims in light of the above-detailed description and still fall within the scope of the present disclosure and claims. In general, in the following claims, the terms used should not be construed to limit the claimed invention to the specific embodiments disclosed in the specification. Accordingly, the claimed invention is not limited by the disclosure, but instead its scope is to be determined entirely by the following claims.

What is claimed is:

1. A processor executed method for predicting diseases in animals, comprising:
receiving patient medical record data;
filtering the received patient medical record data by at least one of species, breed, gender, or geographic location;
separating the filtered patient medical record data into first structured data and unstructured data;
training a first machine learning model on the unstructured data to extract second structured data from the unstructured data;
combining the first structured data and the second structured data to form a training set for a second machine learning model;
training the second machine learning model on the training set formed from the combined structured data to output the likelihood of one or more diseases; and
applying the trained first machine learning model and the trained second machine learning model, in sequence, on new patient medical record data to predict disease diagnosis.

2. The method according to claim 1, wherein the training set for the second machine learning model includes one or more input features extracted from the combined structured data and corresponding ground truth.

3. The method according to claim 2,
wherein the one or more features include one or more of an age of the patient, propensity of the patient to one or more diseases, one or more test results, one or more symptoms, and one or more observations, and
wherein the ground truth includes the likelihood of one or more diseases.

4. The method according to claim 1, wherein the first machine learning model is trained using unsupervised learning.

5. The method according to claim 1, wherein the second machine learning model is trained using supervised learning.

6. The method according to claim 1, further including:
training a plurality of second machine learning models;
evaluating the plurality of second machine learning models using one or more metrics; and
selecting one or more machine learning models of the plurality of second machine learning models for application to the new patient medical record data to predict disease diagnosis.

7. The method according to claim 6, wherein the one or more metrics include prediction error, complexity, explainability, or data size.

8. The method according to claim 1, wherein the first structured data and the second structured data is combined based on data or time information included in the patient medical record data.

9. A clinical diagnostic system comprising:
at least one computer accessible-storage device configured to store instructions; and
at least one processor communicatively connected to the at least one computer accessible storage device and configured to execute the instructions to:
receive patient medical record data;
filter the received patient medical record data by at least one of species, breed, gender, or geographic location;
separate the filtered patient medical record data into first structured data and unstructured data;
train a first machine learning model on the unstructured data to extract second structured data from the unstructured data;
combine the first structured data and the second structured data to form a training set for a second machine learning model;
train the second machine learning model on the training set formed from the combined structured data to output the likelihood of one or more diseases; and
apply the trained first machine learning model and the trained second machine learning model, in sequence, on new patient medical record data to predict disease diagnosis.

10. The system according to claim 9, wherein the training set for the second machine learning model includes one or more input features extracted from the combined structured data and corresponding ground truth.

11. The system according to claim 10,
wherein the one or more features include one or more of an age of the patient, propensity of the patient to one or more diseases, one or more test results, one or more symptoms, and one or more observations, and
wherein the ground truth includes the likelihood of one or more diseases.

12. The system according to claim 9, wherein the first machine learning model is trained using unsupervised learning.

13. The system according to claim 9, wherein the second machine learning model is trained using supervised learning.

14. The system according to claim 9, wherein the at least one processor is further configured to execute the instructions to:
train a plurality of second machine learning models;
evaluate the plurality of second machine learning models using one or more metrics; and
select one or more machine learning models of the plurality of second machine learning models for application to the new patient medical record data to predict disease diagnosis.

15. The system according to claim 14, wherein the one or more metrics include prediction error, complexity, explainability, or data size.

16. The system according to claim 9, wherein the first structured data and the second structured data is combined based on data or time information included in the patient medical record data.

17. A non-transitory computer readable storage medium configured to store a program, executed by a computer, for a clinical diagnostic system, the program including instructions for:
receiving patient medical record data;
filtering the received patient medical record data by at least one of species, breed, gender, or geographic location;
separating the filtered patient medical record data into first structured data and unstructured data;
training a first machine learning model on the unstructured data to extract second structured data from the unstructured data;
combining the first structured data and the second structured data to form a training set for a second machine learning model;
training the second machine learning model on the training set formed from the combined structured data to output the likelihood of one or more diseases; and
applying the trained first machine learning model and the trained second machine learning model, in sequence, on new patient medical record data to predict disease diagnosis.
